(19) United States
(12) Patent Application Publication
(10) Pub. No.: US 2023/0270625 A1
(43) Pub. Date: Aug. 31, 2023

(54) DEVICE FOR THE PHYSICAL RELAXATION OF A PERSON

(71) Applicant: JK-HOLDING GMBH, Windhagen (DE)

(72) Inventor: Jürgen GERSTENMEIER, Neuwied (DE)

(73) Assignee: JK-HOLDING GMBH, Windhagen (DE)

(21) Appl. No.: 18/005,431

(22) PCT Filed: Jul. 14, 2021
(86) PCT No.: PCT/IB2021/056340

§ 371 Date: Jan. 13, 2023

(30) Foreign Application Priority Data
Jul. 15, 2020 (CH) 00879/20

(51) Int. Cl. A61H 37/00; A47C 31/00; A61L 2/10; A61L 9/20

(52) U.S. Cl. CPC A61H 37/00; A47C 31/007; A61L 2/10; A61L 9/20; A61L 2202/11; A61L 2202/14

(57) ABSTRACT

The invention relates to a device for the physical relaxation of a person. The device comprises at least one contact surface (11), on which at least parts of the person come into physical contact with the device. The device additionally comprises at least one relaxation means for generating and/or blocking stimuli in order to relax the person and at least one lighting means (13), which is designed to emit optical radiation in a wavelength range between 200 nm and 230 nm and supply a radiation region with optical radiation with a peak ranging between 207 and 222 nm. The invention also relates to the use of a lighting means for the aforementioned purpose and to a method for disinfecting a device for the physical relaxation of a person.

The present invention relates to a device for the physical relaxation of a human. Furthermore, the invention relates to the use of a light source in such devices for disinfection, and to a method of disinfecting a device for physical relaxation of a human, all in accordance with the respective preambles of the independent claims.

### TECHNOLOGICAL BACKGROUND

In the wake of the pandemic spread of SARS-CoV-2 in 2019 and 2020, public life has experienced numerous restrictions. It is becoming increasingly apparent that some of these restrictions will also have a lasting effect, particularly with respect to behavior in public spaces and social life. One lesson from the Covid 19 pandemic is undoubtedly that in a hypermobile society and high population density in metropolitan areas, it is difficult to prevent the spread of diseases globally. Numerous social measures serve to reduce infection risks in public spaces. Last but not least, however, people's trust in publicly accessible facilities suffers, as these can sometimes carry a certain risk of transmission.

In the short term, measures can be taken on the personnel side to guarantee hygienically impeccable conditions in publicly usable facilities at all times. Ultimately, however, this leads to higher costs in the operation and maintenance of such facilities. Publicly accessible facilities that serve the purpose of relaxation, such as massage devices or relaxation loungers in public spaces, may be particularly affected. Especially in areas where there is a high level of public traffic, it is hardly possible to guarantee a permanent hygienically flawless user surface of relaxation equipment without excessive effort. Particularly affected and critical in this context are, for example, airports, waiting areas, inner city areas, and shopping centers. However, the need for small oases of relaxation is particularly high in turbulent crowds.

Thus, there is a need for devices for the relaxation of a human being, which can be found hygienically impeccable and are highly accepted by consumers, since they always meet the same hygienic standard, regardless of the diligence of a previous user or a cleaning staff.

In terms of the present invention, devices for physical relaxation of a human may particularly be understood to mean devices which can be used by several people in succession. For example, a massage chair placed in a public space, such as a waiting area of an airport, may be the subject matter of the invention. However, specially equipped relaxation and wellness centers can also be included, which, for example, have and offer a plurality of such devices. For the purposes of the present invention, such devices may include, for example, devices selected from the group consisting of: Massage chairs, massage beds, automatically actuated massagers that are stationary or mobile, foot tubs, body tubs, hand baths, relaxation capsules, e.g., with noise-attenuating environments, light baths, steam baths, dry massagers, and sensory deprivation-based relaxation devices.

In common use, such devices are made hygienic mainly by means of disinfectant for subsequent use by another user. However, this presupposes that, on the one hand, the previous user conscientiously performs the disinfection and, on the other hand, a nursing staff does this according to the required standards. In addition, a supply of suitable disinfectants must always be available. Many disinfectants are also aggressive and can dry out or even damage the skin when used. Furthermore, use of a disinfectant includes the manipulation of another object, which in turn can lead to a possible risk of contamination.

It is known that ultraviolet radiation can be used for disinfection. For this purpose, the surface or fluid to be disinfected is exposed to UV radiation, which destroys and inactivates microorganisms. Commonly, radiation at a wavelength of 254 nm is used. If organic compounds are to be broken down as well, wavelengths of less than 200 nm are used. Such wavelengths are known to be harmful to humans. For this reason, light sources with the wavelength ranges mentioned are usually in inaccessible areas, e.g. in vents or in filters, to ensure that contact of the skin with the harmful UV radiation is prevented as far as possible.

### SUMMARY OF THE INVENTION

It is thus an object of the present invention to provide a device of the type mentioned at the outset which overcomes at least one disadvantage of prior art.

Particularly, the object is to provide a device that has a high level of user acceptance in terms of hygienic cleanliness. At the same time, said device should preferably be as efficient as possible in operation and not involve any further personnel costs.

Particularly preferably, the solution according to the invention is suitable for retrofitting existing relaxation devices and making them suitable for the hygienic requirements of the 21st century.

At least one of these objects has been achieved with a device for physical relaxation of a human being according to the characterizing part of the independent claims.

One aspect of the present invention relates to a device for physical relaxation of a human. The device includes at least one contact surface where at least portions of the human come into physical contact with the device.

In terms of the present invention, a contact surface may be, for example, a lying surface or a supporting surface. In most of the devices according to the invention, the person seeking relaxation settles down on the device, in a sitting or lying position, and undergoes a relaxing treatment there. Special devices for physical relaxation of a human may also be provided in which, for example, only parts of the person come into physical contact with the device; these may particularly be foot baths or hand baths.

The device according to the invention further comprises at least one relaxation means for generating and/or inhibiting a stimulus or stimuli for relaxing a human.

In terms of the present invention, a wide variety of relaxation means are suitable for inducing physical relaxation in a human by producing and/or inhibiting a stimulus/stimuli. In its simplest form, the means of relaxation can be a simple, e.g. anatomically adapted, padded couch which is accessible in public spaces and allows for a short “power nap.” Such devices may, for example, be additionally equipped with stimulus-inhibiting devices. For example, darkening hoods or soundproof walls can be provided to reduce auditory and visual stimuli to those seeking relaxation. Relaxation means according to the invention can also provide physically working and driven massage elements, e.g. rotating, pulsating, or vibrating elements which are suitable for targeted relaxing treatment of individual muscle groups or joints. In most devices according to the invention, the relaxation means are additionally provided with pads or supports which make contact with the relaxation means as comfortably as possible for the user. Such relaxation means are known to the skilled person and can be selected and implemented as required. Suitable dry massaging devices are disclosed, for example, in DE 20 2019 105 636 U1.

In a particular embodiment, the contact surface is then at least partially formed by the relaxation means.

The device according to the invention further comprises at least one light source. This light source is configured to emit optical radiation in a wavelength range between 200 nm and 230 nm. In this respect, the light source is further configured to expose a radiation region to optical radiation having a peak in a wavelength range between 207 nm and 222 nm.

Particularly preferably, the radiation area can be aligned in such a way that the contact surface can be substantially completely exposed, particularly so that the contact surface is always exposed to optical radiation in the aforementioned wavelength range if the user is not in the process of contacting it. If the contact surface is currently operated by a user, the optical radiation can also affect the user, for example.

It was surprisingly found that no damage to human skin otherwise expected from UVC radiation occurs in said wavelength range (Longterm effects of 222 nm ultraviolet radiation C sterilizing lamps on mice susceptible to ultraviolet radiation, Yamano, Nozomi et al, Photochemistry and Photobiology, doi: 10.1111/php.13269).

As a particular advantage of the present invention, not only the contact surface, but also a fluid located in the radiation area, such as air or water, is exposed to the disinfecting radiation. Since said radiation in the range according to the invention does not cause any damage to human skin and eyes as otherwise expected from UVC radiation, a particularly safe environment can thus be ensured for a user in a public space, which meets an increased need for safety especially in times of heightened pandemic alert.

The devices according to the invention can thus be set up in public areas without causing any harm to people, and can be operated in a continuous mode. They can always be found in hygienically perfect condition by the user, regardless of the diligence of a cleaning employee. Said UV radiation in the said range is also preferably selected in such a way that at least all surfaces or contact surfaces used in operation can always be exposed to said UV radiation. During operation, the UV lamp with the respective radiation can be switched off or continue to run. In the latter case, the respective surface of the human is also disinfected, which can be an additional desired effect. In addition, the radiation area, which may be defined as an air space between the human and the light source, is also exposed to said radiation, and disinfects it. The solution according to the invention enables a gain in confidence with respect to the use of publicly accessible equipment in public spaces or in fitness centers.

In a particular embodiment, the light source is configured to emit substantially monoenergetic UV radiation having a wavelength peak in the range between 207 and 222 nm and having an energy in the radiation range from at least 0.5 mJ/cm² to at most 500 mJ/cm², particularly between 2 mJ/cm² and 50 mJ/cm², very preferably approx. between 2 mJ/cm² and 20 mJ/cm^{2.}

For example, the energy may be defined as the dose within the radiation area, wherein the radiation area defines a volume, including the beam angles of the light source. Particularly preferably, the light source is designed in such a way that the aforementioned energy in the radiation area is distributed over a beam angle with limb lengths between 0.1 m and 2 m, particularly between 0.5 m and 2 m.

In a particular embodiment, the light source is configured to emit an adjustable energy of substantially monoenergetic UV radiation having a wavelength peak in the range between 207 and 220 nm. Particularly preferably, the light source is designed to provide said energy in a radiation area with said optical radiation via a control system. For example, the energy may be adjustable by a person skilled in the art based on the geometry of the device, the contact area, and/or the radiation area. The energy can also be particularly adjustable on the basis of a specific germ to be combated, wherein the control system is preferably designed in such a way that an adjusted germ or pathogen triggers a particular setting of the energy of the light source for said radiation area.

In particular, the light source is designed to inactivate bacteria from the haemophilus species with UV radiation having a peak in a wavelength range between 207 and 220 nm and an energy from at least 0.5 mJ/cm² to at most 10 mJ/cm² in the radiation range.

In particular, the light source is designed to inactivate viruses of the coronavirus family with UV radiation with a peak in a wavelength range between 207 and 220 nm and an energy from at least 0.3 mJ/cm² to 2 mJ/cm² in the radiation area.

Particularly preferably, the light source is configured to emit substantially monoenergetic UVC radiation with a peak wavelength of 222 nm.

It was surprisingly found that, with the energies mentioned and the respective wavelength range, a high reliability of disinfection of the exposed surface can be achieved, and at the same time the advantages of the respective harmlessness of UVC radiation in the wavelength range mentioned can be achieved.

Without being tied to this theory, the wavelength ranges mentioned seem to be wavelengths that are predominantly absorbed in the skin surface, the cuticle, and which do not succeed in penetrating human cells and cause the undesired cell damage there as other UV radiation elsewhere can cause.

In a particular embodiment, the light source comprises an excimer-based lamp. Particularly, the light source comprises a quasi-monochromatic light source . Alternatively and/or in addition, the light source comprises at least one band-pass filter for reducing an emission spectrum of the light source to a wavelength having a peak in said range, particularly at about 207 nm or 222 nm.

Particularly preferably, the light source comprises a lamp with the excimer molecules selected from the group consisting of: Krypton chlorine or krypton bromine (Kr-Cl, Kr-Br).

In a particular embodiment, the light source comprises a band-pass filter configured to remove wavelengths outside a wavelength in the range between 207 nm and 230 nm. In other words, the band-pass filter is designed to substantially let pass wavelengths shorter than 226 nm. For the purposes of the present invention, substantially letting pass is understood as having an optical transmittance of at least 80%, preferably at least 90%, for optical radiation of the wavelength in question.

In another particular embodiment, the light source comprises a short-pass filter having an optical transmittance of at least 80% for wavelengths shorter than 230 nm. Particularly preferably, the short-pass filter has an edge in a range between 226 and 232 nm. In another particular embodiment, the short-pass filter has an interference filter comprising at least one, preferably two, filter layers.

In a particularly preferred embodiment, the light source comprises an excimer-based lamp that substantially emits light of a wavelength having a peak of 207 nm, particularly a wavelength having a peak of substantially 207 nm at which, at a relative power of 10% or more, the emission spectrum is greater than 200 nm and less than 214 nm, more preferably greater than 204 nm and less than 210 nm.

In an alternative particular embodiment, the light source comprises an excimer-based lamp that substantially emits light of a wavelength having a peak of 222 nm, particularly a wavelength having a peak of substantially 222 nm at which at a relative power of 10% or more the emission spectrum is greater than 215 nm and less than 229 nm, more preferably greater than 219 nm and less than 225 nm.

To achieve the emitted wavelength range, an appropriate dimer pair such as krypton chlorine or krypton bromine may be used and, in a particular embodiment, a suitable band-pass filter may additionally be provided.

In a particular embodiment, the light source according to the invention comprises a cooling system. Particularly preferably, a flow generator is provided at the light source to generate cooling by air.

In a particular embodiment, a heat-conducting structure may be provided to facilitate heat exchange between the lamp and the environment; particularly, surface-expanding fins may be provided to dissipate waste heat. Additionally or alternatively, flow generators such as fans can be installed, which can dissipate an accumulation of heat generated by the waste heat from lamp operation.

In a particular embodiment, the device comprises a plurality of light sources, each having a radiation area.

Particularly in the case of geometrically complex arrangements, such as armchairs or seat pans, it can be advantageous if several light sources are provided, each of which having its own radiation area, thus ensuring that all contact surfaces can be irradiated. It may also be desirable if different radiation areas overlap, e.g., to provide additional radiation to particularly exposed contact surfaces. However, it is also conceivable that a plurality of lamps are installed in parallel, so to speak, and act on the same contact surface, on the one hand to increase a disinfection performance, and on the other hand for structural or design reasons, e.g. to avoid filling the existing spaces with oversized lamps.

In a particular embodiment, the radiation area is configured such that at least one contact area is substantially fully covered in unused condition and exposable to optical radiation having a peak in the range between 207 and 222 nm. Additionally or alternatively, the radiation area can be selected such that, in use, it exposes the person using it in addition to the adjacent contact surfaces.

In a particular embodiment, the light source is configured to emit optical radiation with a peak in the range of about 207 or about 222 nm, with a half-width of about 4 nm.

In another particular embodiment, the arrangement of the light sources according to the invention can be designed in such a way that one state can be transferred into the other, i.e., that the light sources are arranged so as to be movable as a whole.

In a particular embodiment, the device according to the invention comprises a control unit for the at least one light source or for the plurality of light sources, respectively. The control unit can, for example, adjust radiation intensities, intervals, geometric alignment of the radiation areas to respective conditions in each case. For example, the control unit can be designed to carry out a respective maintenance program in which movably arranged light sources carry out a respective sequence that leads to a complete impact on all contact surfaces of the device according to the invention. Likewise, the control unit may be configured to perform appropriate reorientation of the light sources when the device is in use, for example. Particularly preferably, the control unit is designed in such a way that an energy mJ/cm² in the radiation range can be set by the control unit.

In a particular embodiment, the device according to the invention comprises sensors which detect the use of the device, for example. Furthermore, sensors may be provided to detect the body mass of the person using the device. A control unit can then be designed to adjust the radiation applied to the contact surfaces to the body proportions or to the detection of a human being.

In a particular embodiment, the light sources according to the invention comprise a contact protection, which prevents the light sources from being touched by the people using them. In this way, the overall service life of the light sources can be increased and a risk of burning can be minimized. In the simplest embodiment, a grid or a glass pane may be provided, for example, which prevents that the light source is touched.

Since humans are not able to perceive light in the spectra mentioned, the use of the device according to the invention with the respective light sources is not detrimental to the relaxation purpose of the devices shown.

In a special embodiment, the light sources are installed in such a way that they are hardly noticed by the person using the device. For this purpose, suitable screens can be provided, for example, which conceal the light sources.

In a particular embodiment, the device according to the invention comprises carriers for holding the at least one light source. Particularly preferably, the carrier is positioned in such a way that the light source has a beam angle that defines a radiation area in which the contact surface is located substantially completely. Furthermore, a fluid in the radiation area, for example water or air, is exposed to the radiation.

In another particular embodiment, the carrier is designed as a swivel arm and is configured with at least one joint for aligning the radiation area, such that an alignment of the light source can be carried out automatically by a user or by a control unit, which alignment ensures that the beam angle of the light source defines a radiation area in which at least one contact surface is located completely.

Particularly preferably, the carrier can be detachably connected to the device according to the invention.

In a particular embodiment, the support is configured such that the light source is located opposite a contact surface.

Thus, in a particular example of a therapy couch, such as a dry massage table, the light source would be located opposite the couch surface, i.e., above the massage surface. In operation, the light source has a beam angle that covers all four edges of the massage surface substantially completely, such that the contact surface, i.e. the lying surface where the person being treated goes when receiving a massage, is located completely within the exposure region of the radiation area.

In a particular embodiment, the carrier is designed as a robot arm. Particularly preferably, the carrier is designed as a SCARA robot which has at least four axes and four degrees of freedom. In interaction with a control unit, such a robotic arm can, for example, execute a specific maintenance and disinfection program in which, for example, angles and corners are additionally illuminated that are otherwise inaccessible in a static continuous exposure of a special device geometry. The control unit can be designed, for example, to run such a maintenance program before use. If this is done in the presence of a future user, confidence in the hygienic flawlessness of the relaxation device can be further increased. Additionally or alternatively, the control unit may be configured to perform such a maintenance program upon completion of a use of a relaxation device.

In a particular embodiment, the device according to the invention comprises a relaxation means for generating and/or inhibiting stimuli for human relaxation. The relaxation means may be selected from the group consisting of: massage table, therapy chair, dry massager, bath tub, sensory deprivation and/or attenuation capsule, and automatic massager.

Such devices are in principle known to a person skilled in the art. Dry massagers, for example, are devices in which a jet of water transfers kinetic energy to a person to produce a massage effect by placing a flexible membrane between the massage jet and the person. Dry massagers can be equipped similar to waterbeds, for example, which in operation with appropriate nozzles move along the body proportions of the user and thus create a massage effect similar to a massage nozzle in a SPA.

For the purposes of the present invention, sensory deprivation and/or attenuation capsules may be understood as capsules which are substantially sound insulating. In addition, it may be dark inside the capsule. Special sensory deprivation capsules also include a salt brine bath that further creates a feeling of weightlessness. These capsules are usually completely dark. It was surprisingly found that such capsules can ideally be disinfected with the system according to the invention, since disinfection with the UV light in the wavelength ranges mentioned does not interfere with the relaxation effect, since it does not produce visible light for humans. Suitable materials for creating sound-insulating capsules are porous materials, such as melamine sponges, e.g. melamine resin foam and/or foamed materials.

In a particular embodiment, the device according to the invention further comprises a vent for generating a ventilation flow. In the simplest embodiment, a flow generator may for example be provided, which supplies fresh air to a user of a device according to the invention. Additionally, a disinfection chamber may be provided in fluid communication with the ventilation flow. The disinfection chamber may be configured to perform physical disinfection of the ventilation flow.

Since this disinfection chamber can be installed in a closed system to which a user does not have access and said user thus is not exposed to potentially harmful ultraviolet radiation, the disinfection chamber can be based, for example, on a simple UV disinfection lamp with a wavelength of substantially 254 nm.

Preferably, a light trap through which the ventilation flow can pass is constructed around the disinfection chamber, such that air to be disinfected can flow into and out of a disinfection chamber. Fans, for example, can be provided as flow generators.

In a particular embodiment, the device according to the invention comprises a sensor which is particularly designed to detect the position of the light source relative to the contact surface. A suitable sensor would be an infrared sensor, for example. It is also conceivable to provide an accelerometer or a magnetometer that can detect the orientation of the light source in space. A maintenance program adapted to the device can be carried out in interaction with the control unit, for example. In the case of geometrically movable devices according to the invention, the control unit can ensure that all angles and corners are treated by the maintenance program. This may be the case, for example, with a massage table that can be transformed into a chair.

In an additional and/or alternative embodiment, the sensor is configured to detect usage and accordingly define an adjusted beam angle and thus a radiation area that takes into account the possible coverage of the contact surface by a user.

In a particular embodiment, the light source is arranged behind the contact surface, such that the contact surface is irradiated from behind, i.e. through the surface material. This embodiment requires that the contact surface is substantially permeable to the designating radiation in the wavelength range between 200 and 230 nm.

The device according to the invention ensures that, in times of heightened pandemic alert, the confidence of users in publicly accessible or even in private devices for relaxation is maintained. The respective devices can always be kept in a hygienically perfect condition, and there is no need for additional personnel to clean the devices.

It is self-evident to a person skilled in the art that all preferred and special embodiments described can be implemented in any combination in an embodiment according to the invention, provided that they are not mutually exclusive.

Another aspect of the present invention relates to a use of at least one light source configured to emit optical radiation in a wavelength range between 200 nm and 300 nm for generating a radiation area comprising optical radiation having a peak in a wavelength range between 207 nm and 222 nm in a relaxation device, such that at least one contact surface of the relaxation device is located in the radiation area and can be exposed to radiation.

According to the use, existing relaxation devices can be upgraded to a device according to the invention if appropriate light sources can be attached or installed in consideration of the teachings disclosed herein.

In a particular embodiment, the use according to the invention comprises a carrier suitable for functional connection to the relaxation device, such that the radiation area can be aligned relative to the contact surface.

In a particular embodiment, the use according to the invention further comprises connecting interfaces, wherein the light source has respective interfaces and control functions that ensure that the contact surface of the device to be upgraded can be irradiated by the light source. Accordingly, the interface may allow control of the carrier, which may be a robotic arm, for example, by the device.

Another aspect of the present invention relates to a method for disinfecting a device for physical relaxation of a human.

The method comprises the step of providing at least one light source. The light source is designed to emit optical beams with a peak in a wavelength range between 207 nm and 222 nm. For this purpose, the light source has beam angles that define a radiation area. In this radiation area, everything is exposed to light with a peak in a wavelength range between 207 nm and 222 nm. The method according to the invention further comprises the step of positioning the light source. For this purpose, a contact surface of the device to be disinfected must be positioned in a radiation area of the light source. Subsequently, the radiation area is exposed to radiation at the specified wavelength.

In a particular embodiment, the method according to the invention comprises filtering optical radiation in a wavelength range between 200 nm and 230 nm, such that a radiation area can be exposed to optical radiation with a peak in a wavelength range between wavelength range between 207 nm and 222 nm, particularly 207 nm or 222 nm. Particularly preferably, filtering comprises providing a band-pass filter, particularly a short-pass filter having an edge in a wavelength range between 226 nm and 232 nm.

In a particular embodiment, positioning takes place via a control system. For this purpose, a control unit can be provided, for example, which carries out an irradiation as a maintenance program on the basis of the known data relating to the device. In this case, the irradiation can for example be adjusted to the geometric features of the device. For this purpose, a light source can be moved in such a way that the radiation area irradiates different contact surfaces for different lengths of time.

In a particular embodiment, positioning is performed by means of a carrier configured as a swivel arm. This can also be done by the control unit mentioned.

In another particular embodiment, positioning of the light source is performed automatically based on a detected position of the light source relative to a contact surface, e.g., by controlling the light source by a control unit using a swivel arm.

The present invention will be explained in more detail below with reference to specific exemplary embodiments and figures, but without being limited to these. For a person skilled in the art, further advantageous embodiments result from the study of these specific exemplary embodiments. For the sake of simplicity, the same parts are given the same reference numerals in the figures.

### DESCRIPTION OF THE FIGURES

Exemplary embodiments of the invention are described with reference to the following figures. Wherein:

FIG. 1 shows an embodiment of a device according to the invention.

FIG. 2 shows an alternative embodiment of a device according to the invention;

FIG. 3a shows a component set for upgrading an existing physical relaxation device;

FIG. 3b shows a detailed view of the light source of device 3a;

FIG. 4 shows another alternative embodiment of the device according to the invention, and

FIG. 5 shows another embodiment of a device according to the invention;

FIG. 6 shows a transmission curve of a suitable band-pass filter, and

FIG. 7 shows an embodiment of a wavelength range according to the invention.

### DETAILED DESCRIPTION OF THE INVENTION

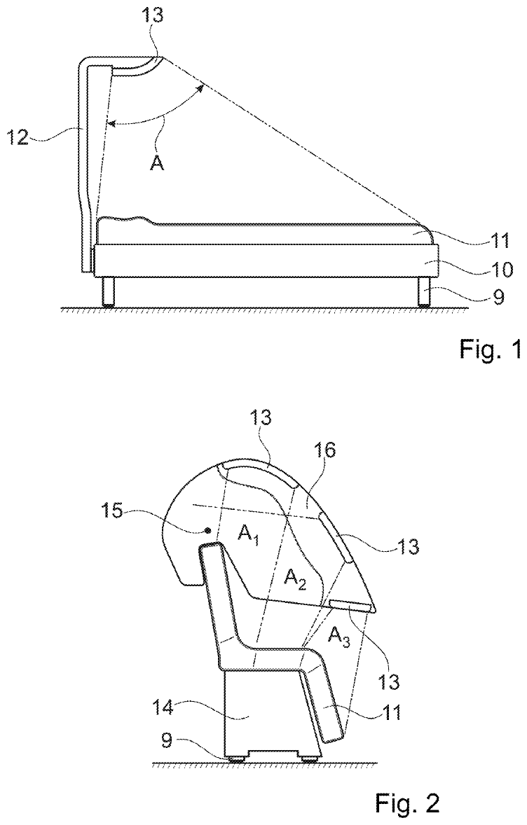

FIG. 1 shows a basic embodiment of a device according to the invention. The device is a device for physical relaxation of a person, in which the person seeking relaxation can assume a lying position. A contact surface 11 is provided for this purpose, which is designed as a lying surface. In the present example, the lying surface is anatomically shaped such that a head area has a support function at the neck. The contact surface 11 can particularly be provided as a massage surface. Although not shown in FIG. 1, the contact surface 11 can, for example, be configured as a membrane which is capable of transferring to the user kinetic forces transmitted from a couch body 10 to the contact surface 11. Thus, the couch body 10 may be formed as a dry massage device together with a contact surface 11 configured as a membrane.

The dry massage device comprises a series of pivotable water jet nozzles and a common space filled with water between the couch neck body 10 and the contact surface 11 formed as a membrane. The massage jets can selectively massage individual areas of the body of a person lying on the device for physical relaxation with a jet of water. The membrane prevents the water jet from wetting the user in the process. Appropriate pumps inside the couch body 10 can provide a steady flow of water. In addition, such devices for physical relaxation of a person can be designed with heating and/or cooling elements to heat, respectively cool, the respective water jet. The contact surface, which is configured as a membrane, allows this temperature effect to act on the user.

In use, the contact surface 11 is exposed to direct body contact by the user. A person who wants to use the device shown for physical relaxation lies down on the contact surface 11. Thus, it is crucial that this surface is found as hygienically flawless as possible. For this purpose, the device shown in FIG. 1 has a light source 13 with a beam angle that defines a radiation area A. The beam angle is selected and controlled by the shape of the lamp and any reflectors in the lamp body in such a way that the contact surface is located in the radiation area from the head end to the foot end and over the entire width of the contact surface and can be exposed to the respective optical radiation. In the present example, the light source is connected to the couch body 10 via a carrier 12. The couch body 10 can be positioned on a surface by means of feet 9.

In particular embodiments, the feet 9 may further comprise rollers to make the devices movable.

In the present example, the light source is selected to emit UV radiation at a wavelength of 222 nm. To ensure that the wavelength is within as narrow a spectrum as possible, with a peak at 222 nm, a narrow spectrum krypton chlorine excimer lamp is used in this example. In addition, the lamp is designed with a band-pass filter that substantially absorbs wavelengths outside 222 nm. For example, short-pass filters made of synthetic quartz glass with single or multiple coatings are suitable.

In operation, the light source can act continuously. Any safety limits and maximum doses can be set. As a rule, such devices are in any case limited in their duration of use. For example, devices accessible in public spaces for the relaxation of a person are often equipped with a payment system with which the respective user purchases a certain unit of time for use on the device. Preferably, a non-contact payment system is used for a device according to the invention. A typical treatment on a massage table according to the invention, e.g. the dry massage table described at the outset, lasts between five and 45 minutes. Due to the low effects of the wavelengths mentioned for the human body as well as for the human eye, and its effectiveness regarding microorganisms, the respective light source does not need to be switched off during use. On the contrary, it may even be desirable if, in addition to the massage effect, a disinfecting effect of the user's clothing or body also takes place.

In addition, in the present example, air cooling may be provided for the krypton chlorine gas lamp with a peak at 222 nm.

Another embodiment according to the invention of the device is shown in FIG. 2. FIG. 2 can be used, for example, as an inhaler and steam hood in which an inhalation treatment is performed while the user is seated. The capsule shown in FIG. 2 can also be used as a relaxation capsule, e.g. by using sensory deprivation materials such as insulating materials.

Accordingly, the device for relaxing a person shown in FIG. 2 has a contact surface 11 in the form of a seating surface. This contact surface 11 is based on a chair body 14, which can also be placed on the ground by means of feet 9. A hood 16 which extends over the head and shoulders to the middle of a user's torso is provided in the head area of the contact surface 11, on which a person seeking relaxation can sit down. The hood can be configured to be movable, so that it can be transferred from an open (not shown) to a closed state (shown) via a swivel mechanism. This makes it easier for people to climb on the device. Inside the hood is a defined hood volume. The hood can be opened and closed via a pivot axis 15. Some devices of the type shown are mechanically capable of opening and closing the hood 16. For example, after a selected program, the hood can be closed automatically and a respective relaxation program can run. Devices shown may have a variety of possible relaxation programs. For example, the hood 16 may be lined or formed with noise attenuating materials, such as acoustic foams, such that noise attenuation occurs internally and relaxation is possible through auditory deprivation. It is also conceivable that additional loudspeakers are provided inside the hood to play an appropriate music or speech program, such as a guided meditation. The material of the hood 16 may be transparent, opaque or non-transparent, depending on the desired type of relaxation. Also, outlet nozzles may be provided for vapors and odorous substances, which create a relaxing atmosphere inside the hood 16 by supplying such substances to a user's respiration. Also conceivable are various light programs, which can promote relaxation.

In a particular embodiment, for example, the hood and hood interior may be configured to play a light program corresponding to a forest spectrum. At the same time, relaxing forest sounds, such as the rustling of leaves or the chirping of birds, can be played, and relaxation-promoting scents and substances such as resin and pine scents can be fed into the hood via an outlet.

In the present example, the contact surface 11 is not a planar surface, but is divided into different areas. Accordingly, a plurality of light sources 13 have been provided on the hood, each having its own radiation area A1, A2, A3. The reflectors and lamp geometry for radiation areas are selected such that essentially the entire contact surface 11 can be exposed to the respective radiation. In particularly used areas, e.g. in the area of a headrest (not shown), two light sources can form an overlapping area between the radiation areas A1 and A2 to even increase the disinfection effect. For efficiency reasons, in the present example, one light source 13 is then sufficient in less critical areas, such as the foot area, to form a radiation area A3 that disinfects the foot area. As in the device shown above according to FIG. 1, the light sources do not need to be switched off in this embodiment as well when they are used by humans, since the light sources 13 comprise lamps capable of emitting a spectrum with a peak of 207 nm.

Appropriate band-pass filters can further narrow the wavelength range so that the peak becomes more specific and the corresponding adverse effects of UV radiation on the body are avoided without diminishing the disinfecting effect of the UV light. In the present example, a krypton bromine excimer lamp is used to generate a 207 nm UV light wavelength.

Such lamps are known with respect to their mode of operation (Buonanno M., et al. 207-nm UV Light—A Promising Tool for Safe Low-Cost Reduction of Surgical Site Infections; In Vitro Studies. PLoS One 8(10), 2013).

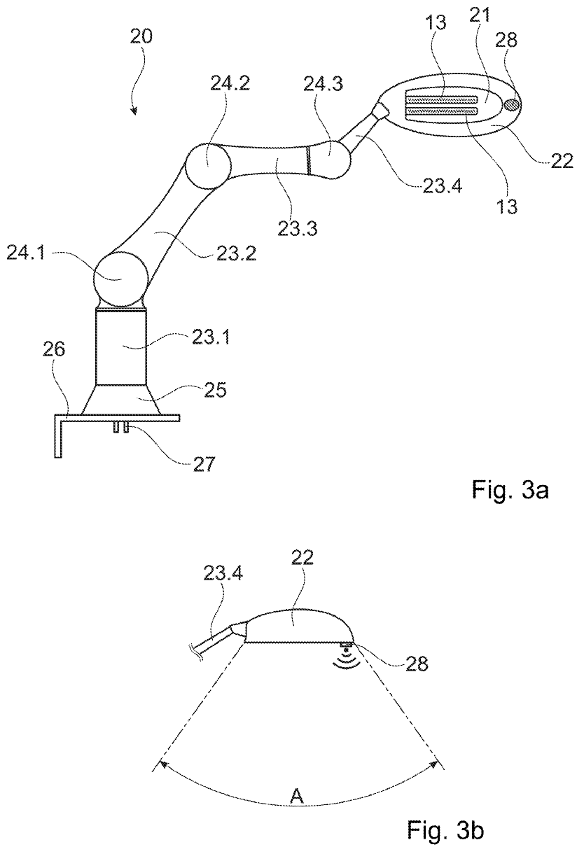

In order to further improve the disinfection of contact surfaces and to upgrade existing devices for physical relaxation, FIG. 3a shows a device which comprises a respective light source and can be connected to a carrier with a device for physical relaxation. The disinfection device 20 includes, at a head end, a respective light source 13 comprising an excimer lamp capable of generating optical radiation having a peak in a wavelength range between 207 and 222 nm. An energy in the radiation area of approx. 2-20 mJ/cm² is targeted.

The light source 13 is housed in a light source socket and has an additional reflector screen 21, which controls a respective radiation cone. The reflector 21 and the light sources 13 are housed in a lamp housing 22. Preferably, the lamp housing 22 comprises means for facilitating heat dissipation, for example, ribs or fins may be provided which allow for easier heat exchange between the “placeholder” 22 and the ambient temperature. The light source 13 and the respective lamp socket form the distal end of a pivot arm. Also attached to the distal end of the “placeholder” 22 is a sensor 28 which is capable of detecting optical signals. For example, the sensor may be configured to detect motion or to detect the presence of an obstacle or user.

The optical sensor can also be configured to measure a distance. In addition, accelerometers and magnetometers (not shown) may be provided in the lamp head 22 which detect the position of the lamp head in space. The beam may be formed of various joint modules 23.1, 23.2, 23.3 and 23.4, which are connected to each other by various joints 24.1, 24.2, 24.3. The number of joint modules 23.1, . . . 23.4 and joints 24.1, 24.2, 24.3 can be formed by the desired number of degrees of freedom and the alignment geometry of the carrier.

The carrier rests on a foot element 25, which is coupled to a connecting plate 26. An interface 27 allows the device to be connected to a control system and to an electrical supply of a device for physical relaxation of a human. If said device has a respective control unit, a respective maintenance program can be carried out by the carrier and the disinfection arm, for example, by means of a data record containing the device identification and type, as well as the configuration of the device, which program ensures that the entire contact surface is treatable. For example, difficult-to-reach zones of a device for relaxation or generally of a device that can be used by the public can be reached by means of the robot, e.g., recessed grips or levers can be specifically addressed by the robot arm and irradiated with the light source.

Thus, the present embodiment is not limited to static disinfection of a static contact surface, but may perform dynamic disinfection after use, during use, or prior to use of a device for relaxation. In this case, the sensor system can be used to prevent that a user is unintentionally affected by a maintenance program in that the robot arm detects such a user and keeps an appropriate safety distance. Preferably, the light source 13 is protected from being touched by a user via contact protection.

The interface 27 may further be configured to be connected to a ventilation system. A respective ventilation flow can thus also be directed through the robot arm and ejected, for example, via respective ejection nozzles at the distal end, i.e. at the head part of the robot arm. Particularly preferably, such a ventilation system is also designed with a disinfection chamber. Since this ventilation system can include a disinfection chamber that is not in contact with the user, it can be operated with conventional disinfection, e.g., with a wavelength range of 254 nm. Thus, a device for the relaxation of a human can be generated which is safe to operate and ensures virtually germ-free use, even in public areas.

FIG. 3b shows the respective distal end, i.e. the head part of the robot arm of FIG. 3a in detail. Accordingly, the lamp housing 22 together with the accordingly mounted reflectors and the light source (not shown in FIG. 3b) form a respective radiation area A. This radiation area A can be fed as a parameter into a control unit, such that the control unit is able to use this radiation area to ensure that proper disinfection of all surfaces can take place. In fact, the control unit can guarantee that a maximum effective distance is always maintained between the light source 13 and the contact surface 11 to be disinfected. For support, the sensor 28 can dynamically detect the respective distance and forward it to the control unit.

The pivotable elements 23.4 allow numerous degrees of freedom, making it possible to o disinfect an object essentially independently of even complicated geometries with the solution according to the invention.

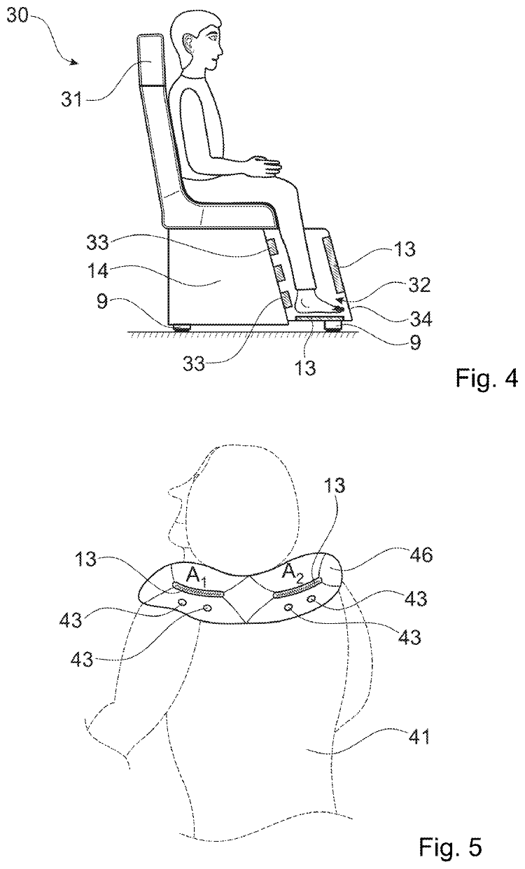

Another embodiment is shown in FIG. 4 . The device shown is a footbath device 30. The footbath device 30 also has a seat back 31 and a seat body 14, which rests on said feet 9 already described and can thus be positioned on a surface. A new feature of the footbath device 30 is a foot tub 32 which is filled with a liquid. A plurality of massage nozzles 33 which kinetically apply a jet of liquid to the lower legs and calves are formed on the side facing the seat body 14. The foot tub 34 also has two light sources 13. The light sources are positioned so that substantially the entire interior of the tub 34 can be exposed to the optical radiation.

Surprisingly, it was found that water is in no way detrimental to the mode of operation of the respective UV light in the spectrum mentioned as a transmission medium. This also ensures that the water used for the footbath device 30 is always germ-free. In accordance with the embodiments described above, the light sources 13 may emit a wavelength with a peak in the range of 207 to 222 nm, wherein one wavelength may be selected or a combination of a plurality of wavelengths and respective band-pass filters may be provided to keep the peak narrow.

FIG. 5 shows another device according to the invention, which in this case is a wearable vest with a neck portion 46. This neck portion 46 has mechanically operating massage rollers 43 which kinetically work the neck muscles and counteract relaxation of the user 41. On the body side (for illustrative purposes, the neck element 46 is shown transparent in the present example), illuminating means 13 are provided which cover the contact surface between the user 41 and the rolling rollers 43 with respective radiation areas A1, A2, such that the entire contact surface between the neck device 46 and the user 41 can be disinfected.

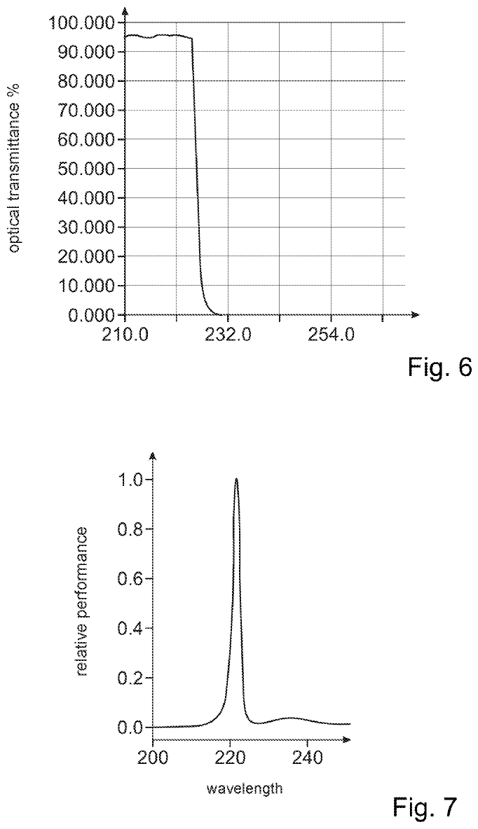

FIG. 6 shows a transmission curve of a suitable filter for a light source, e.g. as used in the embodiment according to FIG. 1. The filter used is a filter based on a double-coated synthetic quartz glass, e.g. made of SiCl₄ (silicon tetrachloride). The coating serves as an interference filter and can be applied by means of a physical or chemical gas vapor deposition process. Particularly preferably, the filters used have a sharp transition between transmission and reflection.

The exemplary short-pass filter shown has an optical transmission of more than 90% in the range between 210 and about 230 nm, with the edge at 229 to 232 nm.

FIG. 7 shows an example of a generated wavelength for use according to the invention with a peak in the range of 222 nm. Such a wavelength can be generated when using a krypton chlorine excimer lamp with an emission spectrum with a peak at 222 nm after filtering with a filter as described for FIG. 7.

The relative power is essentially in the range of 222 nm, with a half-width of the spectrum of about 4 nm in the present example.

The present invention discloses devices and the use of light sources, as well as methods of operating said devices, which are suitable for improving public confidence in relaxation devices while providing hygienic devices that counteract the spread of germs.

It goes without saying that numerous other areas of application in the field of relaxation devices are conceivable to a person skilled in the art on the basis of the exemplary embodiments described.

What is claimed:

1. A device for physical relaxation of a human, comprising:
a. at least one contact surface on which at least parts of the human come into physical contact with the device;
b. at least one relaxation means for producing and/or inhibiting a stimulus/stimuli to relax the human; and
c. at least one light source adapted to emit optical radiation in a wavelength range between 200 nm and 230 nm, and wherein said at least one light source is adapted to irradiate a radiation area with optical radiation having a peak in a wavelength range between 207 nm and 222 nm.

2. The device according to claim 1, wherein said light source is adapted to expose said radiation area to optical radiation having a peak in a wavelength range between 207 nm and 222 nm, such that within said radiation area there is a dose between at least 0.5 mJ/cm² to at most 450 mJ/cm².

3. The device according to claim 1, wherein the light source comprises an excimer-based lamp.

4. The device according to claim 1, wherein the light source comprises a band-pass filter adapted to substantially let pass therethrough wavelengths within a spectral range between 200 nm and 230 nm.

5. The device according to claim 4, wherein the band-pass filter is a short-pass filter having a transmission range of less than 230 nm.

6. The device according to claim 1, wherein the light source comprises a cooling system, particularly comprises a flow generator for cooling by air.

7. The device according to claim 1, wherein the device comprises a plurality of light sources, each having a radiation area.

8. The device according to claim 1, wherein the radiation area is adapted to substantially completely cover the at least one contact surface in the unused state.

9. The device according to claim 1, comprising a control unit for the light source(s).

10. The device according to claim 1, comprising a carrier for holding the at least one light source, particularly wherein the carrier is configured as a swivel arm with at least one joint for aligning the radiation area.

11. The device according to claim 1, wherein the relaxation device for producing and/or inhibiting a stimulus/stimuli for human relaxation is a relaxation device selected from the group consisting of: massage table, therapy chair, dry massager, bath tub, sensory deprivation and/or attenuation capsule, and automatic massager.

12. The device according to claim 1, further comprising a ventilation system for generating a ventilation flow, and at least one disinfection chamber in fluid communication of the ventilation flow and adapted to perform physical disinfection of the ventilation flow.

13. The device according to claim 1, further comprising a sensor adapted to detect the position of the light source relative to the contact surface.

14. A method of using at least one light source, which is designed to emit optical radiation in a wavelength range between 200 nm and 230 nm, for generating a radiation area with optical radiation having a peak in a wavelength range between 207 nm and 222 nm in a relaxation device, such that a contact surface of the relaxation device is irradiated in the effective range of the radiation area.

15. The method according to claim 14, wherein a carrier is provided for functional connection to the relaxation device, such that the radiation area can be aligned relative to the contract surface.

16. A method of disinfecting a device for physical relaxation of a human, comprising the steps of:
a. providing at least one light source adapted to emit optical radiation in a wavelength range between 200 nm and 230 nm for exposing a radiation area to optical radiation having a peak in a wavelength range between 207 nm and 222 nm;
b. positioning the light source so that at least one contact surface of the device to be disinfected is located in a radiation area; and
c. exposure of the surface to radiation with a peak in a wavelength range between 207 nm and 222 nm.

17. The method according to claim 16, wherein the positioning is performed via a controller.

18. The method according to claim 16, wherein the positioning is performed by means of a carrier designed as a swivel arm.

19. The method according to claim 16, wherein the positioning is performed automatically by a control unit based on a detected position of the light source relative to the contact surface.

20. The device according to claim 1, wherein said light source is adapted to expose said radiation area to optical radiation having a peak in a wavelength range between 207 nm and 222 nm, such that within said radiation area there is a dose between at least 2 mJ/cm² to at most 20 mJ/cm².
